(19) United States
(12) Patent Application Publication
(10) Pub. No.: US 2009/0287075 A1
(43) Pub. Date: Nov. 19, 2009

(54) BIOLOGICAL SIGNAL MEASUREMENT SENSOR

(75) Inventors: Hiroshi KUBOTA, Tokyo (JP); Sunao TAKEDA, Tokyo (JP)

Correspondence Address: KIMBLE INTELLECTUAL PROPERTY LAW, PLLC, 1701 PENNSYLVANIA AVE., NW, SUITE 300, WASHINGTON, DC, 20006, US

(73) Assignee: NIHON KOHDEN CORPORATION, Tokyo (JP)

(21) Appl. No.: 12/407,839

(22) Filed: Mar. 20, 2009

(30) Foreign Application Priority Data
Mar. 21, 2008 (JP) 2008-073036

(51) Int. Cl. A61B 5/04

(52) U.S. Cl. 600/391

(57) ABSTRACT

A biological signal measurement sensor is to be fixed to a living body with an adhesive. The biological signal measurement sensor includes: a surface, which is to be in contact with a part of the living body; and a first portion, which is opposite to the surface and to which the adhesive is to be attached. At least a part of the first portion has a first patterned indented shape.

The present invention relates to a biological signal measurement sensor for measuring a biological signal.

A noninvasive biological signal measurement sensor is attached to the body surface. In order to stably perform a measurement, an adhesive tape is often applied onto the sensor to enhance the fixation of an attached portion.

As the adhesive tape, an adhesive tape which is suitable for a living body is used. In order to reduce dermal irritation, such as skin irritation, caused by abrasion of cornified layer, the bonding area of an adhesive tape is widened while suppressing the adhesive force of an adhesive agent as far as possible, whereby the adhesive tape is improved so as to maintain the adhesive force at an adequate degree. Therefore, a soft gel-like adhesive agent is often used.

When an adhesive tape is used for a long-term, the adhesive force between the bonding surface of the sensor and the adhesive agent is sometimes made stronger than that between the tape base material and the adhesive agent by invasion of the adhesive agent to the boding surface of the sensor. The use of a soft gel-like adhesive agent may cause a problem of so-called “adhesive deposit” that, when the sensor is peeled off, the adhesive agent remains on the boding surface of the sensor, i.e., the back surface and peripheral portion of the sensor.

The adhesive deposit produces “sticky sense” to give a sense of discomfort to the sensor attached person to which a work of attaching the sensor is performed. In each sensor attachment, moreover, the back surface and peripheral portion of the sensor must be cleaned with using disinfectant alcohol or the like, thereby complicating the work of the sensor attaching person who performs the work of attaching the sensor.

In the case of a reusable sensor, also for the purpose of preventing interpatient infection from occurring, the sensor must be always kept clean. Therefore, it is a significant problem to prevent adhesive deposit from occurring.

There is a related-art method of solving the problem of adhesive deposit, in which, as in “release paper” which is used in an adhesive sheet or the like, the adhesive force is suppressed by coating the sheet with a silicone resin or fluorine resin that has a low surface energy. In this related-art method, however, additional costs of the coating material and a process of the material are required. In a biological signal measurement sensor of the high-mix low-volume production type, this is problematic from the standpoint of cost.

Furthermore, the following related arts are known in which the application surface of a label is improved so that, when the label is applied to a plastic container or the like, the label can be easily peeled off (see JP-A-8-198224, JP-A-2001-219936 and JP-A-2007-84096).

The related art disclosed in JP-A-8-198224 relates to application of a label. In order to make a peripheral portion of a label to be hardly peeled off, a region which exhibits a strong adhesive force (strong-force region) is disposed in at least a part of the outer circumferential region of the bonding surface of the label. The adhesive force is weakened in the region inside the strong-force region, thereby causing the label to be easily peeled off.

In order to cope with the problem of adhesive deposit of the sensor, however, the adhesive force must be suppressed in the whole of the bonding surface of the sensor (the back surface and peripheral portion of the sensor).

The region where the adhesive force is weak is characterized in that the region has an embossed pattern in which the surface roughness is defined to be 10.5 μm≦Rz≦11.8 μm and 0.154 μm≦Sm≦0.219 μm. As described above, however, a soft gel-like adhesive agent is often used in an adhesive tape, and hence an embossed pattern of the above-mentioned conditions is not expected to exert the effect of reducing the bonding area on such a tape.

The related art disclosed in JP-A-2001-219936 relates a plastic container having a surface to which a label is to be applied, and is characterized in that a region which exhibits a strong adhesive force is disposed in a label peripheral portion in the same manner as JP-A-8-198224. Therefore, the related art is not appropriate to solve the problem of adhesive deposit of the sensor.

The related art disclosed in JP-A-2007-34096 relates a plastic container having a surface to which a label is to be applied, and provides an improvement in which dimensions of convex portions and a patterned indented section shape are defined and a label can be peeled off more easily. However, the surface area of the back surface and peripheral portion of a biological signal measurement sensor is significantly smaller than the label-to-be applied surface which is proposed in the related art disclosed in JP-A-2007-84096. Therefore, the defined dimensions of convex portions cannot form concaves and convexes in the back surface and peripheral portion of the sensor, and hence the related art is not useful.

### SUMMARY

It is therefore an object of the present invention to provide a biological signal measurement sensor in which the shapes of the back surface and the peripheral portion of the sensor are improved so as to solve the problem of adhesive deposit, and which benefits both the sensor attaching person and the patient (sensor attached parson).

In order to achieve the object, according to the invention, there is provided a biological signal measurement sensor which is to be fixed to a living body with an adhesive, the biological signal measurement sensor comprising:

a surface, which is to be in contact with a part of the living body; and

a first portion, which is opposite to the surface and to which the adhesive is to be attached, wherein

at least a part of the first portion has a first patterned indented shape.

The biological signal measurement sensor may further include: a inclined second portion, arranged around the first portion. At least a part of the second portion may have a second patterned indented shape.

The first patterned indented shape and the second patterned indented shape may be different from each other.

The first patterned indented shape and the second patterned indented shape may include at least one of a circular shape, a square shape, a rectangular shape, a triangular shape and a polygonal shape.

Following relationships may be satisfied: (a surface area of the part of the first portion having the first patterned indented shape, which is to be in contact with the adhesive)≦α×(a surface area which is to be in contact with the adhesive in case where the first portion does not have the first patterned indented shape); and 0<α<1. The α is a characteristic coefficient of the adhesive.

The first portion may have one of a planar shape and a curved shape.

### BRIEF DESCRIPTION OF THE DRAWINGS

FIG. 1 is a view showing a first example of a patterned indented shape formed in a portion of the biological signal measurement sensor of the present invention which is to be in contact with an adhesive agent of an adhesive tape.

FIG. 2 is a view showing a second example of the patterned indented shape formed in a portion of the biological signal measurement sensor of the present invention which is to be in contact with an adhesive agent of an adhesive tape.

FIGS. 3A and 3B are diagrams showing a state where the biological signal measurement sensor is fixed to a part of a living body with an adhesive tape.

FIG. 4 is a view showing a example of the biological signal measurement sensor of the invention.

### DETAILED DESCRIPTION OF PREFERRED EMBODIMENTS

In the present invention, therefore, the sensor is configured so that concaves and convexes (patterned indented shapes) are formed in the back surface and peripheral portion of the sensor, whereby the bonding area between the sensor and an adhesive agent is intentionally reduced, and, even after a long-term application, the adhesive force between the bonding surface of the sensor and the adhesive agent is not stronger than that between a tape base material and the adhesive agent, and adhesive deposit does not occur.

In the invention, the sensor is realized only by adding the patterned indented shapes to the back surface and the peripheral portion of the sensor. In the design of molds for a sensor portion, the patterned indented shapes can be formed into the molds. Unless the patterned indented shapes are special ones, therefore, the sensor can be realized without requiring an additional cost. The production cost can be reduced as compared with the above-described related-art coating method.

In the invention, in order to solve the problem of adhesive deposit on the back surface and peripheral portion of the sensor, as described above, the sensor is configured so that concaves and convexes (patterned indented shapes) are formed in the back surface and peripheral portion of the sensor, whereby the bonding area between the sensor and the adhesive agent is intentionally reduced, and, even after a long-term attachment, the adhesive force between the bonding surface of the sensor and the adhesive agent is not stronger than that between a tape base material and the adhesive agent, and adhesive deposit does not occur.

The shapes of the concaves and convexes are not particularly restricted. However, the concaves and convexes must be designed so that the surface areas of the convex portions (the bonding surface of the sensor) satisfy the following relationship. In the viewpoints of the design and the aesthetic, preferably, concaves and convexes of orderly shapes are disposed in a portion where a relatively flat surface can be formed (for example, the back surface of the sensor).

(Total of surface areas of convex portions which are to be in contact with adhesive tape)≦α×(surface area which is to be in contact with adhesive tape in case where there are no concave and convex portions)

(where α is a characteristic coefficient which is determined by an adhesive agent of the used adhesive tape, and 0<α<1).

In the case where, for example, a biological signal measurement sensor which is shown in FIG. 4 has a length of 20 mm and a width of 30 mm, at least an opposing portion (a portion being opposite to a surface of the sensor which is to be in contact with a part of living body) and inclined portion of the sensor are formed by an ABS resin, and a medical tape which includes a PVC foam material as a base material and an acrylic adhesive agent is applied, when the above-mentioned α is set to α=0.4, contamination by the adhesive agent of the foam tape occurs at a very low degree.

In a sensor, the peripheral portion may have a three-dimensionally curved surface structure. In such a case, it is necessary to form thereon concaves and convexes having a pattern which is different from that of concaves and convexes formed on the opposing portion.

Also in this case, the shapes of the concaves and convexes are not particularly restricted, but the whole shape thereof must satisfy the above expression.

### EXAMPLES

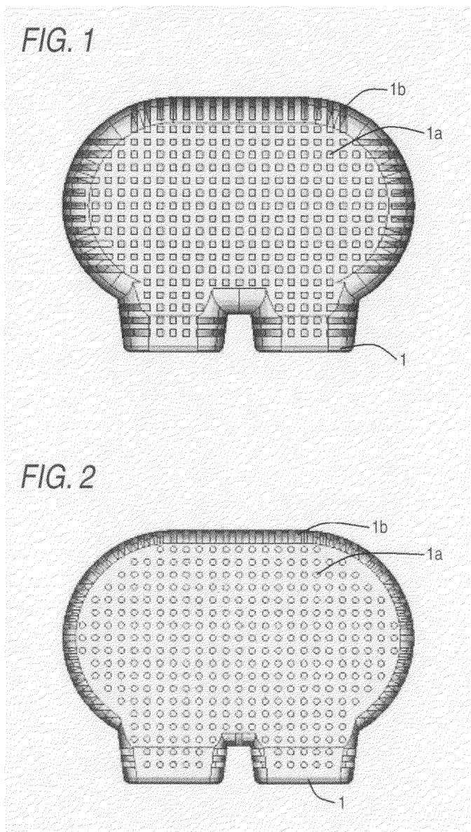

Next, examples will be described with reference to FIGS. 1 to 3.

FIG. 1 is a view showing a first example of the patterned indented shape formed in a portion of the biological signal measurement sensor which is to be in contact with an adhesive agent of an adhesive tape.

In FIG. 1, 1 denotes a biological signal measurement sensor in which a patterned indented shape including concaves and convexes is formed in an opposing portion 1a that is opposite to the surface to be in contact with a part of a living body, and that is planar or curved, and a peripheral portion 1b. In the patterned indented shape, square convexes are evenly formed in the opposing portion 1a, and rectangular convexes are evenly formed in the peripheral portion 1b.

FIG. 2 is a view showing a second example of the patterned indented shape formed in the portion of the biological signal measurement sensor which is to be in contact with an adhesive agent of an adhesive tape.

In FIG. 2, 1 denotes a biological signal measurement sensor in which a patterned indented shape is formed in an opposing portion 1a that is opposite to the surface to be in contact with a part of a living body, and that is planar or curved, and a peripheral portion 1b. In the patterned indented shape, circular convexes are evenly formed in the opposing portion 1a, and rectangular convexes and triangular convexes are evenly formed in the peripheral portion 1b.

In FIGS. 1 and 2, the convexes are evenly formed in each of the surfaces of the opposing and peripheral portions. Alternatively, the convexes may be formed in a part of each of the surfaces.

The convexes which are formed in each of the surfaces are not required to have the same shape, and may have different shapes such as a circle, a square, a rectangle, a triangle, a polygon, or a combination of the shapes.

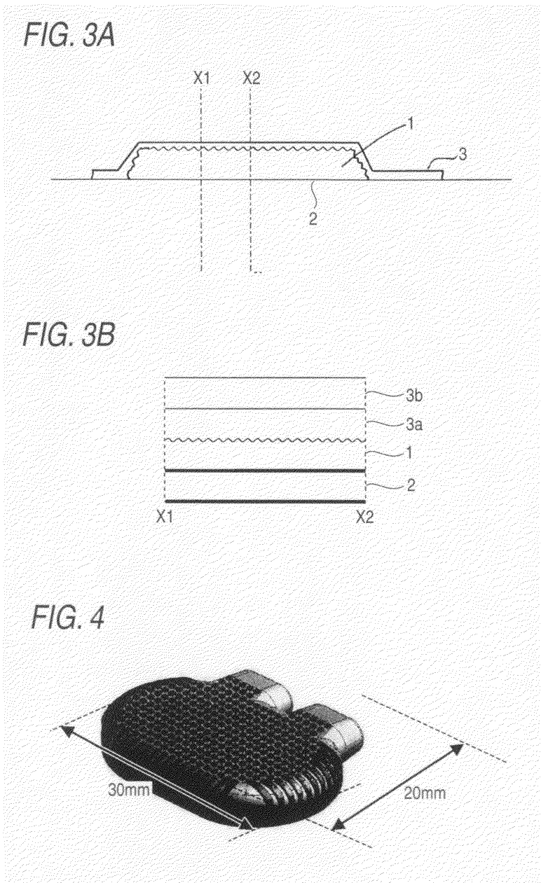

Next, the state where the biological signal measurement sensor of the invention is fixed to a part of the living body with an adhesive tape will be described with reference to FIG. 3.

As shown in FIG. 3A, an attachment surface (surface including portions for emitting and receiving measurement light) of the biological signal measurement sensor 1 is placed on the part of the living body 2, and the sensor is fixed by the adhesive tape 3 from the upper side.

FIG. 3B is a diagram enlargedly showing the portion between lines X1 and X2 in FIG. 3A. The biological signal measurement sensor 1, an adhesive agent layer 3a of the adhesive tape 3, and a base material 3b of the adhesive tape 3 are formed as plural stacked layers on the living body 2. In the contacting faces of the biological signal measurement sensor 1 and the adhesive tape 3, as described above, the upper face (the opposing portion 1a and the peripheral portion 1b) of the biological signal measurement sensor 1 is formed into a patterned indented shape. After the adhesive tape is peeled off, therefore, the remaining amount of the paste (adhesive agent) is so small that the sensor attached person does not feel “sticky sense” and a sense of discomfort is not given to the sensor attached person. The work of, in each sensor attachment, cleaning the back surface and peripheral portion of the sensor with using disinfectant alcohol or the like becomes unnecessary, whereby the work of the sensor attaching person is prevented from being complicated.

In the case of a reusable sensor, also for the purpose of preventing interpatient infection from occurring, the sensor must be always kept clean. According to the invention, adhesive deposit does not occur.

According to an aspect of the invention, it is possible to realize a biological signal measurement sensor in which the shapes of the back surface and peripheral portion of the sensor are improved so as to solve the problem of “adhesive deposit” due to an adhesive tape, and which benefits both the sensor attaching person and the patient (sensor attached person).

The sensor is realized only by adding the patterned indented shapes to the back surface and peripheral portion of the sensor. In the design of molds for a sensor portion, the patterned indented shapes can be formed into the molds. Unless the patterned indented shapes are special ones, therefore, the sensor can be realized without requiring an additional cost. In a biological signal measurement sensor of the high-mix low-volume production type, the production cost can be reduced as compared with the related-art method where a sensor is coated with a silicone resin or a fluorine resin.

What is claimed is:

1. A biological signal measurement sensor which is to be fixed to a living body with an adhesive, the biological signal measurement sensor comprising:
a surface, which is to be in contact with a part of the living body; and
a first portion, which is opposite to the surface and to which the adhesive is to be attached,
wherein at least a part of the first portion has a first patterned indented shape.

2. The biological signal measurement sensor according to claim 1, further comprising:
a inclined second portion, arranged around the first portion,
wherein at least a part of the second portion has a second patterned indented shape.

3. The biological signal measurement sensor according to claim 2, wherein
the first patterned indented shape and the second patterned indented shape are different from each other.

4. The biological signal measurement sensor according to claim 2, wherein
the first patterned indented shape and the second patterned indented shape include at least one of a circular shape, a square shape, a rectangular shape, a triangular shape and a polygonal shape.

5. The biological signal measurement sensor according to claim 3, wherein
the first patterned indented shape and the second patterned indented shape include at least one of a circular shape, a square shape, a rectangular shape, a triangular shape and a polygonal shape.

6. The biological signal measurement sensor according to claim 1, wherein
following relationships are satisfied:
(a surface area of the part of the first portion having the first patterned indented shape, which is to be in contact with the adhesive)≦α×(a surface area which is to be in contact with the adhesive in case where the first portion does not have the first patterned indented shape); and
0<α<1, and
the α is a characteristic coefficient of the adhesive.

7. The biological signal measurement sensor according to claim 1, wherein
the first portion has one of a planar shape and a curved shape.
